(19) United States
(12) Patent Application Publication
(10) Pub. No.: US 2021/0128838 A1
(43) Pub. Date: May 6, 2021

(54) CARTRIDGE HOLDER AND MEDICINE INJECTION DEVICE COMPRSING THE SAME

(71) Applicant: PHILOSYS CO., LTD., Gunsan-si (KR)

(72) Inventors: Woo Hyek CHOI, Yongin-si (KR); Jeong Sik KIM, Seoul (KR)

(21) Appl. No.: 17/030,336

(22) Filed: Sep. 23, 2020

(30) Foreign Application Priority Data
Oct. 31, 2019 (KR) 10-2019-137756

(51) Int. Cl. A61M 5/24

(52) U.S. Cl. CPC A61M 5/2422; A61M 2205/583

(57) ABSTRACT

According to an embodiment, there are provided a cartridge holder and a medicine injection device including the cartridge holder. The cartridge holder comprises a holder case having an insertion space in which a cartridge is inserted, a cross section of the holder case, perpendicular to a lengthwise direction thereof, having a ring shape and a cartridge support connected to the holder case, the cartridge support supporting an outer surface of the medicine cartridge inserted in the insertion space to fix the medicine cartridge. The cartridge support may allow a cross sectional center of the medicine cartridge to match a cross sectional center of the holder case and aligns the medicine cartridge, with the medicine cartridge placed in the insertion space.

### CROSS-REFERENCE TO RELATED APPLICATIONS

This application claims priority to Korean Patent Application No. 10-2019-137756 filed in the Korean Intellectual Property Office on Oct. 31, 2019, the disclosure of which is incorporated by reference herein in its entirety.

### TECHNICAL FIELD

Embodiments of the disclosure relate to a cartridge holder and a medicine injection device having the cartridge holder.

### DISCUSSION OF RELATED ART

Conventionally, insulin-dependent patients measure blood sugar on an empty stomach, after lunch, before or after dinner, and before sleep using a self-blood sugar tester.

When blood sugar control is needed, the patient determines and injects an appropriate amount of insulin on her own using an insulin syringe.

Such blood sugar management requires a self-blood sugar tester, insulin cartridge, or insulin injection device.

The patient accurately records the measured glucose level and the amount of insulin injected and shows the record to a specialist to receive feedback.

For example, an insulin-dependent patient can check the current blood sugar level through a self-measurement device by dropping blood collected by piercing the skin through a lancet and dropping it on a test strip.

When blood sugar control is determined to be necessary, the patient may use an insulin injector.

The patient manually sets the amount of insulin recommended by her doctor on the insulin injector.

The patient places the insulin injector on the skin to be injected and manually injects the insulin.

The patient herself is required to record the measured blood glucose level and the injected amount.

### SUMMARY

According to an embodiment, there are provided a cartridge holder compatible with various standards of medicine cartridges and a medicine injection device including the cartridge holder.

According to an embodiment, there are provided a cartridge holder easy to couple by aligning a medicine cartridge in a proper position while simultaneously inserting the medicine cartridge and a medicine injection device including the cartridge holder.

There is provided a cartridge holder to which a medicine cartridge is coupled. The cartridge holder comprises a holder case having an insertion space in which a cartridge is inserted, a cross section of the holder case, perpendicular to a lengthwise direction thereof, having a ring shape and a cartridge support connected to the holder case, the cartridge support supporting an outer surface of the medicine cartridge inserted in the insertion space to fix the medicine cartridge.

According to an embodiment, there is provided a cartridge holder to which a medicine cartridge is coupled. The cartridge holder comprises a holder case having an insertion space in which a cartridge is inserted, a cross section of the holder case, perpendicular to a lengthwise direction thereof, having a ring shape and a cartridge support connected to the holder case, the cartridge support supporting an outer surface of the medicine cartridge inserted in the insertion space to fix the medicine cartridge.

The cartridge support may allow a cross sectional center of the medicine cartridge to match a cross sectional center of the holder case and aligns the medicine cartridge, with the medicine cartridge placed in the insertion space.

The cartridge support may include a support member disposed between an inner circumferential surface of the holder case and an outer circumferential surface of the medicine cartridge. The support member may apply an external force to the outer surface of the medicine cartridge.

At cross section view perpendicular to the lengthwise direction of the holder case, the support member may protrude from the inner circumferential surface of the holder case to the cross sectional center of the holder case and contact the medicine cartridge to adjust a distance from the cross sectional center of the holder case.

The holder case may include a holder slot formed through a surface thereof. The support member may be connected to the holder case to be positioned in the holder slot.

A plurality of support members may be connected to the holder case. At cross section view perpendicular to the lengthwise direction of the holder case, the plurality of support members may be spaced apart from each other at equal intervals.

The cartridge holder may further comprise a cartridge guide inserted in the insertion space and surrounding an outer circumferential surface of the medicine cartridge. The support member may be connected to the cartridge guide.

The cartridge guide may include a plurality of guide slots formed through a surface thereof and arranged at equal intervals at cross section view perpendicular to the lengthwise direction thereof. A plurality of support members may be placed in the plurality of guide slots. Each of the support members may apply an external force in a direction of aligning the medicine cartridge via an elastic force.

The holder case may include an insertion groove formed in an outer surface along a circumferential direction thereof and a plurality of through holes arranged at equal intervals along the insertion groove and formed through the holder case. The cartridge support may include a ring-shaped ring member fitted to the insertion groove and a plurality of support protrusions connected to the ring member and protruding to a receiving space of the holder case via the through holes.

The support protrusions may be formed of a compressible elastic material. The support protrusions may apply an elastic force to the medicine cartridge, with the medicine cartridge placed in the receiving space.

According to an embodiment, a medicine injection device comprises a cartridge holder including a holder case including an insertion space in which a medicine-containing medicine cartridge is inserted and a cartridge support placed in the insertion space and applying an external force to an outer surface of the medicine cartridge to thereby align a position of the medicine cartridge, a needle unit including a needle for injecting the medicine to a user's body, and a driver connected with the cartridge holder and applying a pressure to the medicine cartridge to discharge the medicine from the medicine cartridge to the needle.

The cartridge support may include a support member to apply an external force to the medicine cartridge to allow a cross sectional center of the medicine cartridge to be positioned coaxially with a cross sectional center of the holder case.

While the medicine cartridge is inserted in the insertion space, the support member may store elastic energy as going away from the cross sectional center of the holder case and apply the external force to the outer surface of the medicine cartridge by the stored energy.

A plurality of support members may be formed at equal intervals at cross section view of the medicine cartridge. The support members may contact the outer surface of the medicine cartridge.

According to an embodiment, the cartridge holder and the medicine injection device including the cartridge holder are compatible with various standards of medicine cartridges, allowing for universal use

According to an embodiment, the cartridge holder and the medicine injection device including the cartridge holder may align a medicine cartridge in a proper position simultaneously with insertion of the medicine cartridge, thereby allowing for an easier coupling of the medicine cartridge.

According to an embodiment, the cartridge holder and the medicine injection device including the cartridge holder are not limited to the foregoing effects and other various effects may be apparent to one of ordinary skill in the art from the following description of embodiments.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a view illustrating an example of using a medicine injection device according to an embodiment;

FIG. 2 is a perspective view illustrating a medicine injection device according to an embodiment;

FIG. 3 is a view illustrating an internal configuration of a medicine injection device according to an embodiment;

FIG. 4 is a perspective view illustrating a cartridge holder according to an embodiment;

FIG. 5 is a front view illustrating a cartridge holder according to an embodiment;

FIG. 6 is a perspective cross-sectional view of the cartridge holder, taken along line VI-VI of FIG. 5;

FIG. 7 is a cross-sectional view of the cartridge holder, taken along line VI-VI of FIG. 5;

FIG. 8 is a front view illustrating a cartridge holder according to an embodiment;

FIG. 9 is a front view illustrating a medicine cartridge according to an embodiment;

FIG. 10 is a perspective view illustrating a cartridge support according to an embodiment;

FIG. 11 is a cross-sectional view of a cartridge holder, taken along line XI-XI of FIG. 8;

FIG. 12 is a perspective view illustrating a cartridge holder according to an embodiment;

FIG. 13 is a perspective view illustrating a cartridge guide according to an embodiment; and

FIG. 14 is a perspective cross-sectional view taken along line XIV-XIV of FIG. 12.

### DETAILED DESCRIPTION OF EXEMPLARY EMBODIMENTS Hereinafter, embodiments of the disclosure are described in detail with reference to the accompanying drawings.

The same or substantially the same reference denotations are used to refer to the same or substantially the same elements throughout the specification and the drawings.

When determined to make the subject matter of the present disclosure unclear, the detailed description of the known configurations or functions may be skipped.

Such denotations as “first,” “second,” “A,” “B,” “(a),” and “(b),” may be used in describing the components of the present disclosure.

These denotations are provided merely to distinguish a component from another, and the essence of the components is not limited by the denotations in light of order or sequence.

When a component is described as “connected,” “coupled,” or “linked” to another component, the component may be directly connected or linked to the other component, but it should also be appreciated that other components may be “connected,” “coupled,” or “linked” between the components.

The same reference denotations may be used to refer to the same or substantially the same elements throughout the specification and the drawings.

Unless stated otherwise, the description of any one embodiment may be applied to other embodiments of the disclosure, and no repetitive or duplicate description is given of the same or substantially the same elements or features.

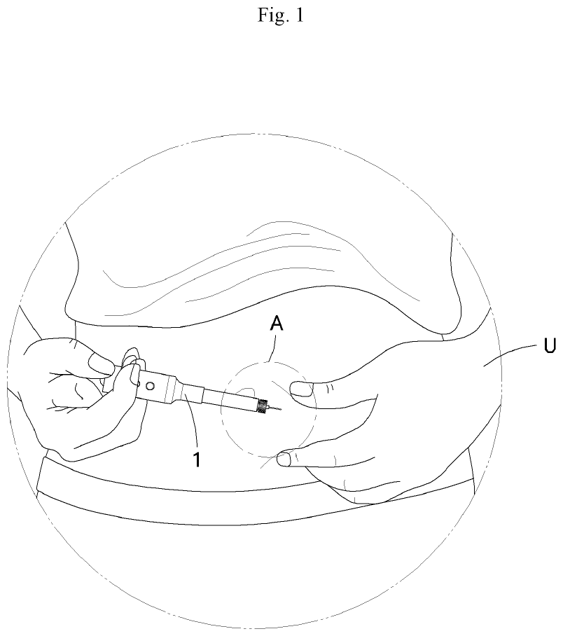

FIG. 1 is a view illustrating an example of using a medicine injection device according to an embodiment.

Referring to FIG. 1, according to an embodiment, a medicine injection device 1 may be used to sense or obtain information about a biological material in the user U's body and to inject a medicine into the user U's body.

The medicine injection device 1 may be used in an invasive manner in which the medicine injection device 1 penetrates the skin of the user U and is inserted into the body.

Hereinafter, the skin area of the user U where the medicine injection device 1 is inserted is referred to as an “invasion area A.”

The medicine injection device 1 may be inserted into the invasion area A and inject a target medicine to the body of the user U.

For example, the target medicine injected to the body of the user U via the medicine injection device 1 may be insulin.

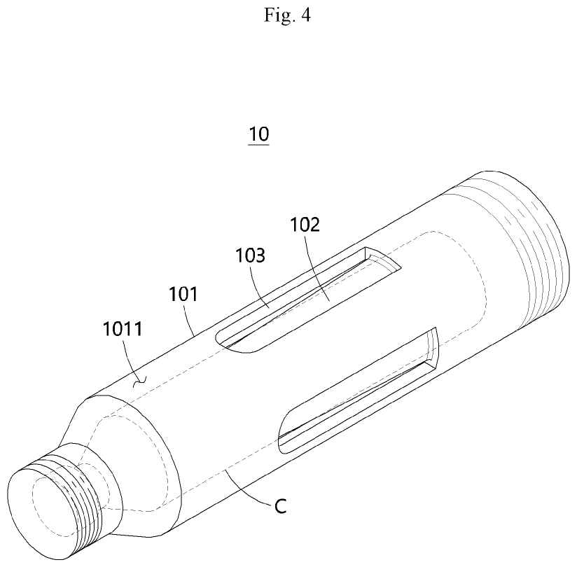

A medicine cartridge C (refer to FIG. 4) may be replaceably inserted to the medicine injection device 1.

In this case, various standards of medicine cartridges may be used in the medicine injection device 1.

In other words, the medicine injection device 1 may have a high degree of compatibility regardless of the standards of medicine cartridges.

This is described below.

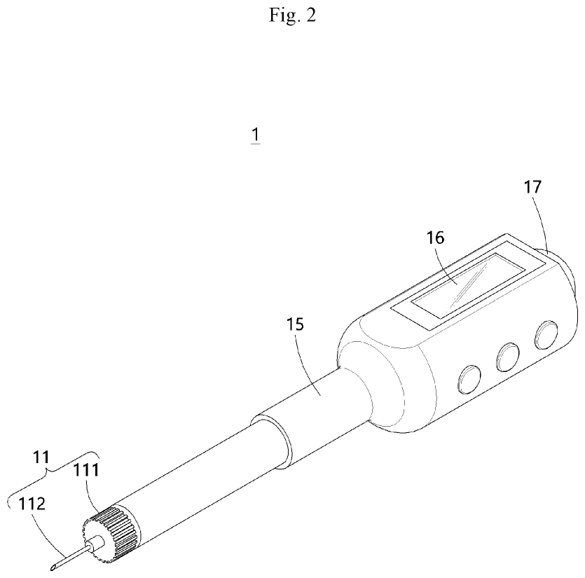

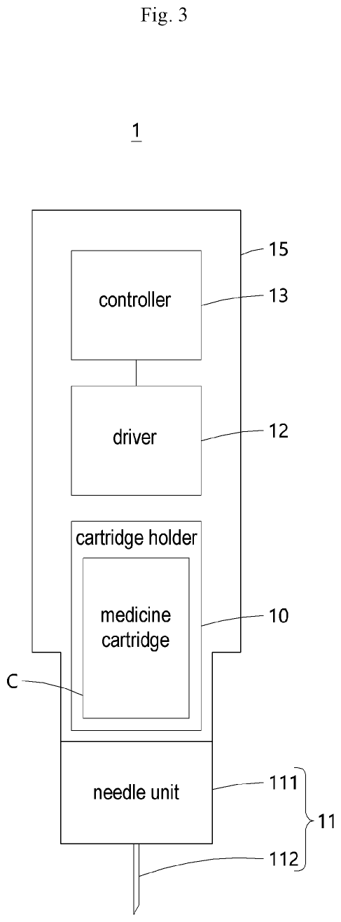

FIG. 2 is a perspective view illustrating a medicine injection device according to an embodiment. FIG. 3 is a view illustrating an internal configuration of a medicine injection device according to an embodiment.

Referring to FIGS. 2 and 3, according to an embodiment, the medicine injection device 1 may include a needle unit 11, a body housing 15, a display unit 16, an operation unit 17, a cartridge holder 10, a driver 12, an analyzer, and a controller 13.

The needle unit 11 may be inserted into the invasion area.

The needle unit 11 may be inserted into the body of the user U and deliver the medicine through the invasion area to the body of the user U.

The needle unit 11 may also suck in the user's body fluids.

Specifically, as the medicine injection device 1 is inserted into the invasion area A and sucks in the user's body fluid, the medicine injection device 1 may sense information about the biological material in the body of the user U.

The biological material may be an element, e.g., glucose, in the body fluid.

The needle unit 11 may be detachably coupled to the body housing 15.

In other words, the needle unit 11 may be selectively coupled to the body of the medicine injection device 1 and, as necessary, is replaceable.

However, this is merely an example. For example, the needle unit 11 may be integrated with the body housing 15.

For ease of description, an example is described below in which the needle unit 11 is detachably coupled with the body housing 15.

The needle unit 11 may include a support 111 connected to the body housing 15 and a needle 112 projecting from the support 111 in the lengthwise direction thereof.

The support 111 may support the needle 112.

The support 111 may be detachably coupled to the body housing 15.

For example, the support 111 may be connected to the cartridge holder 10.

The support 111 may include a structure that may be coupled to the body housing 15.

For example, the support 111 may include a connector that may be electrically connected with the analyzer and the controller 13.

This is not described in detail.

The needle 112 may be directly inserted into the user's body.

The needle 112 may project from the support 111 in the lengthwise direction thereof and may have a sharp tip to penetrate the user's skin.

The needle 112 may include a hollow formed therein in the lengthwise direction thereof.

The medicine in a medicine cartridge C may be injected into the user's body through the hollow.

The needle unit 11 may be removed from the medicine injection device. Thus, as the needle unit 11 needs to be replaced, e.g., for sanitary purposes, the needle unit 11 may be separated and removed from the body housing 15.

Thus, in the medicine injection device, the needle unit 11 is easily replaceable, so that easier maintenance and long-term use are possible for the medicine injection device 1.

The display unit 16 is disposed on an outer surface of the body housing 15 and may display information about the medicine injection device 1 to the user.

For example, the display unit may display information related to the target biological material analyzed via the medicine injection device or information related to the operation of the medicine injection device.

For example, the user may check the glucose level or the amount of insulin injected, via the display unit 16.

The display unit 16 may display the information to the user, in various manners, such as by text, symbols, or graphs.

The operation unit 17 may be disposed on an outer surface of the body housing 15 and be manipulated, controlled, or operated by the user.

The operation unit 17 may be operated by the user's control action and transfer operation information to the controller 13.

The cartridge holder 10 may include an internal space in which the medicine cartridge C is inserted.

The cartridge holder 10 may be coupled to the medicine cartridge C inserted therein.

With the medicine cartridge C coupled to the cartridge holder 10, the medicine cartridge C may discharge the medicine to the needle unit 11 as the medicine injection device operates.

The driver 12 may apply a pressure to the medicine cartridge C to allow the medicine to be discharged out of the medicine cartridge C.

The driver 12 may be connected with the cartridge holder 10 and apply an external force to a side of the medicine cartridge C coupled to the cartridge holder 10 to thereby discharge the medicine from the medicine cartridge C to the needle unit 11.

The analyzer may analyze the user's biometric information or biological information.

The analyzer may be electrically connected with the needle unit 11 and may analyze the obtained target biological material information, e.g., information about the glucose level in the user's body.

The analyzer may determine the amount of the medicine that needs to be injected to the body of the user U, based on the analyzed biological material information.

For example, the analyzer may determine an amount of insulin, appropriate for the user, based on the measured user glucose level.

The analyzer may determine the amount of insulin to be injected, via an algorithm for the relationship between glucose level and insulin or may determine an appropriate amount of insulin to be injected, based on data received an external server, e.g., a medical history for the user U.

However, this is merely an example, and the process of determining the amount of medicine to be injected to the user's body via the analyzer may be determined in other various manners.

The controller 13 may control the operation of the medicine injection device 1.

The controller 13 may control the operation of the driver 12 according to the amount of medicine to be injected, determined by the analyzer, thereby adjusting the amount of medicine discharged from the medicine cartridge C to the needle 112.

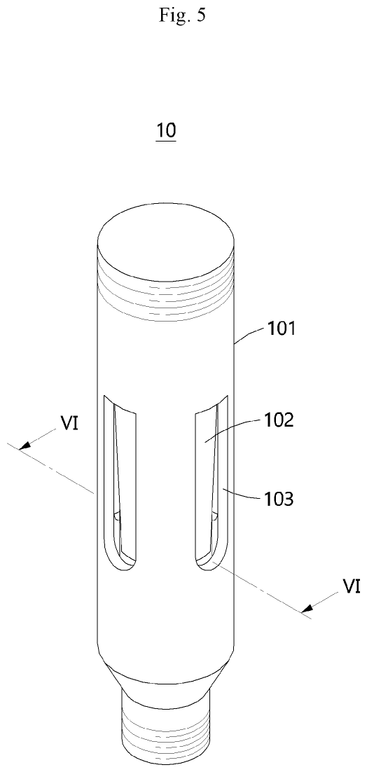

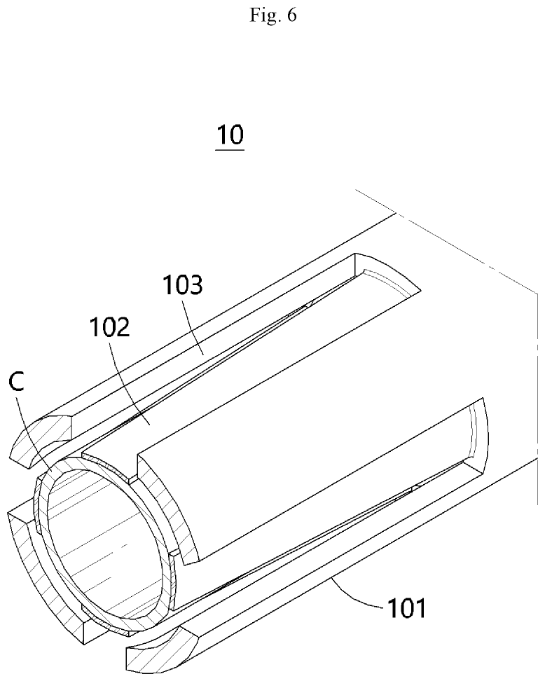

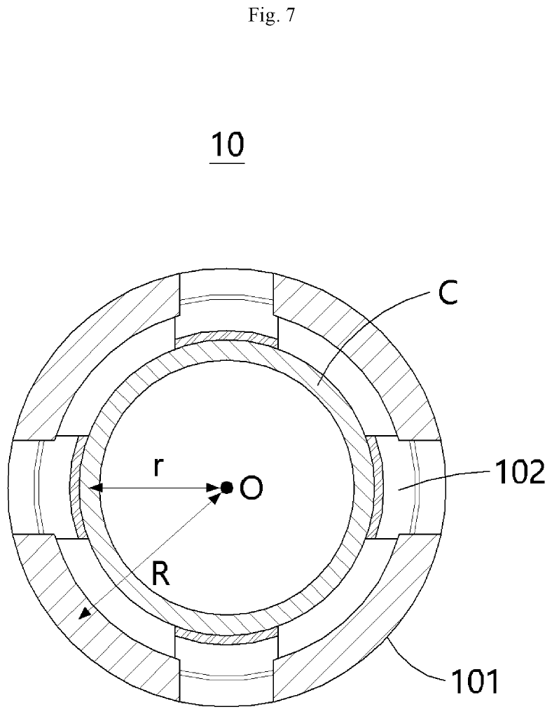

FIG. 4 is a perspective view illustrating a cartridge holder according to an embodiment. FIG. 5 is a front view illustrating a cartridge holder according to an embodiment. FIG. 6 is a perspective cross-sectional view of the cartridge holder, taken along line VI-VI of FIG. 5. FIG. 7 is a cross-sectional view of the cartridge holder, taken along line VI-VI of FIG. 5.

Referring to FIGS. 4 to 7, the cartridge holder 10 may be coupled to the medicine cartridge C.

The cartridge holder 10 may allow the medicine cartridge C to be inserted therein and may align the position of the inserted medicine cartridge C.

With the medicine cartridge C inserted in the cartridge holder 10, the medicine cartridge C may be aligned in a proper couplable position regardless of its standard or specifications.

Thus, the cartridge holder 10 may secure a high degree of compatibility so that various standards of medicine cartridges may be used.

The cartridge holder 10 may include a holder case 101 and a cartridge guide.

The holder case 101 may include an insertion space 1011 formed therein along the lengthwise direction thereof.

The medicine cartridge C may be inserted in the insertion space 1011 of the holder case 101.

The holder case 101 may be formed so that the cross section perpendicular to the lengthwise direction has a ring shape.

At cross-section view, the inner diameter of the holder case 101 may be not less than the outer diameter of the medicine cartridge C.

The cartridge support may align the position of the medicine cartridge C inserted in the insertion space 1011 of the holder case 101.

To couple the medicine cartridge C in the medicine injection device, the center O of the cross section perpendicular to the lengthwise direction of the medicine cartridge C needs to match the center O of the cross section perpendicular to the lengthwise direction of the holder case 101.

The cartridge support may be connected to the holder case 101 and support the outer surface of the medicine cartridge C inserted in the insertion space 1011.

The cartridge support may apply an external force to the outer surface of the medicine cartridge C so that the center O of the cross section perpendicular to the lengthwise direction of the medicine cartridge C matches the center O of the cross section perpendicular to the lengthwise direction of the holder case 101.

In other words, the medicine cartridge C may be aligned in the insertion space 1011 of the holder case 101 by being supported by the cartridge support.

The cartridge support may include a support member 102 disposed between the inner circumferential surface of the holder case 101 and the outer circumferential surface of the medicine cartridge C.

As the support member 102 is connected to the inner circumferential surface of the holder case 101 and supports the outer circumferential surface of the medicine cartridge C, the support member 102 may fix the position of the medicine cartridge C in the insertion space 1011 in the aligned state and prevent the medicine cartridge C from moving in the insertion space 1011.

The support member 102 may protrude from the inner circumferential surface of the holder case 101 to the center O of the cross section perpendicular to the lengthwise direction of the holder case 101.

With the support member 102 contacting the outer surface of the medicine cartridge C, the distance from the center O of the cross section perpendicular to the lengthwise direction of the holder case 101 may be adjusted.

In other words, the distance between the support member 102 and the center O of the cross section perpendicular to the lengthwise direction of the holder case 101 may be varied depending on the outer diameter of the medicine cartridge C.

By the structure, the support member 102 may apply an external force while supporting the outer surface of the medicine cartridge C regardless of the standard or specifications of the medicine cartridge C, thereby fixing the medicine cartridge C in the aligned position.

With the medicine cartridge C aligned, the center O of the cross section of the medicine cartridge C may match the center O of the cross section of the holder case 101 as shown in FIG. 7.

The distance r from the center O of the cross section to the outer circumferential surface of the medicine cartridge C may be shorter than the distance R from the center O of the cross section to the inner circumferential surface of the holder case 101.

In this case, the support member 102 may support the outer circumferential surface of the medicine cartridge C, thereby preventing the medicine cartridge C from moving inside the holder case 101.

Holder slots 103 may be formed to penetrate the surface of the holder case 101.

In other words, the holder slots 103 may be formed to penetrate the outer surface and inner surface of the holder case 101.

The holder slot 103 may have a lengthwise direction parallel with the lengthwise direction of the holder case 101.

An end of the support member 102 may be connected to the holder case 101, with the support member 102 positioned in the holder slot 103.

In other words, the support member 102 and the holder case 101 may be integrally formed with each other and, with no external force applied, the support member 102 may project inside the holder case 101.

Where the medicine cartridge C is inserted in the insertion space 1011, the support member 102 may support the outer surface of the medicine cartridge C and receive an external force in the circumferential direction of the holder case 101.

As pushed away in the circumferential direction, the support member 102 may store elastic energy and pressurize the medicine cartridge C to the center of the cross section of the holder case 101 by the stored elastic energy.

A plurality of support members 102 may be connected to the holder case 101.

The plurality of support members 102 may be arranged at equal intervals when viewed form above the cross section perpendicular to the lengthwise direction of the holder case 101.

In other words, the plurality of support members 102 may support different portions of the outer surface of the medicine cartridge C, in the state where the medicine cartridge C has been inserted in the insertion space 1011.

The portions supported by the plurality of support members 102 may be formed at equal angles from the center of the cross section of the medicine cartridge C.

For example, there may be provided four support members 102 as shown in FIG. 6, and the four support members 102 may be arranged at the intervals of 90° on the outer surface of the medicine cartridge C.

By the structure, the plurality of support members 102 simultaneously pressurize the outer surface of the medicine cartridge C and, thus, the pressurizing forces of the plurality of support members 102 may be canceled off in the state where the center of the cross section of the medicine cartridge C is positioned at the center of the cross section of the holder case 101.

Thus, the position of the medicine cartridge C may be stably aligned.

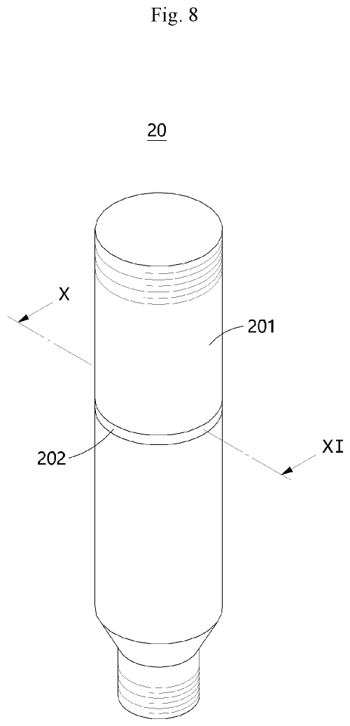

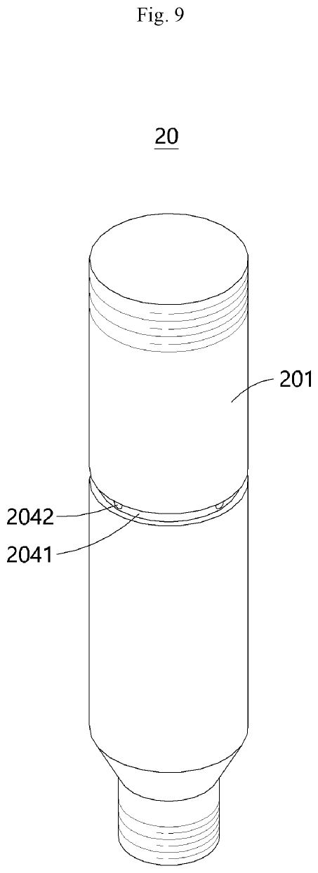

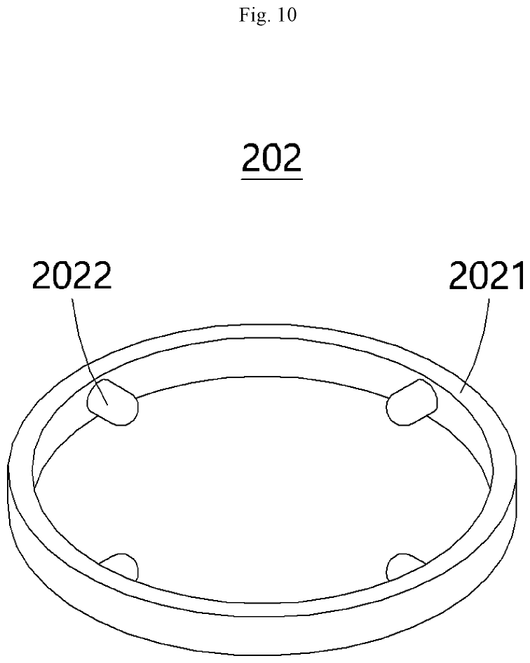

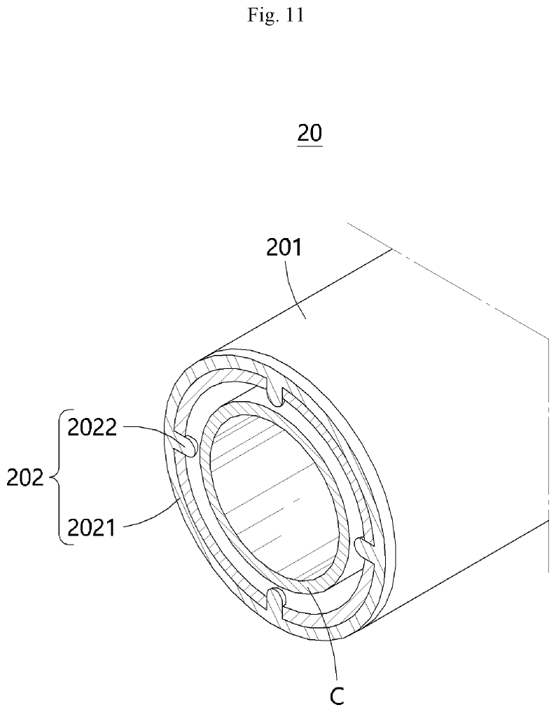

FIG. 8 is a front view illustrating a cartridge holder according to an embodiment. FIG. 9 is a front view illustrating a medicine cartridge according to an embodiment. FIG. 10 is a perspective view illustrating a cartridge support according to an embodiment. FIG. 11 is a cross-sectional view of a cartridge holder, taken along line XI-XI of FIG. 8.

Referring to FIGS. 8 to 11, according to an embodiment, the cartridge holder 20 may include a holder case 201 and a cartridge support 202.

The holder case 201 may include an insertion space 2011 formed in the lengthwise direction thereof and in which the medicine cartridge C is inserted.

An insertion groove 2041 may be formed, along the circumferential direction, in the outer surface of the holder case 201.

The insertion groove 2041 may be depressed to a predetermined depth along the circumferential direction of the holder case 201.

A plurality of through holes 2042 may be formed in the insertion groove 2041.

The plurality of through holes 2042 may be formed inside the insertion groove 2041 and may be arranged at equal intervals.

For example, four through holes 2042 may be formed as shown in FIG. 10, and the four through holes 2042 may be arranged at the intervals of 90° at the cross section view of the holder case 201.

The cartridge support 202 may include a ring-shaped ring member 2021 and a plurality of support protrusions 2022 protruding from the ring member 2021 to the center of the ring member 2021.

The ring member 2021 may be fitted into the insertion groove 2041 to surround the circumference of the holder case 201.

The ring member 2021 may have a thickness corresponding to the depth of the insertion groove 2041.

In this case, with the ring member 2021 fitted into the insertion groove 2041, the outer circumferential surface of the ring member 2021 and the outer circumferential surface of the holder case 201 may not be stepped from each other.

With the ring member 2021 fitted into the insertion groove 2041, the plurality of support protrusions 2022 may be inserted to the plurality of through holes 2042.

The support protrusions 2022 inserted to the through holes 2042 may project to the receiving space of the holder case 201.

As described above, in the case where four through holes 2042 are formed, and the four through holes 2042 are arranged at the intervals of 90° at the cross section view of the holder case 201, the ring member 2021 also has four support protrusions 2022, and the support protrusions 2022 may be inserted to the through holes 2042.

The support protrusions 2022 may be formed of a compressible elastic material.

With the medicine cartridge C inserted in the insertion space, the support protrusions 2022 may contact the outer circumferential surface of the medicine cartridge C.

The support protrusions 2022 are compressed in the circumferential direction of the holder case 201 by the medicine cartridge C, supporting the outer circumferential surface of the medicine cartridge C.

The support protrusions 2022 may pressurize the medicine cartridge C to the center of the cross section of the holder case 201 while filling the gap between the medicine cartridge C and the holder case 201.

By the structure, the cartridge guide may support the outer circumferential surface of the medicine cartridge C and align the medicine cartridge C regardless of the standard or specifications of the medicine cartridge C inserted to the insertion space of the holder case 201.

In other words, since the support protrusions 2022 are compressible, although the outer diameter of the medicine cartridge C increases, the support protrusions 2022 may stably support the outer circumferential surface of the medicine cartridge C.

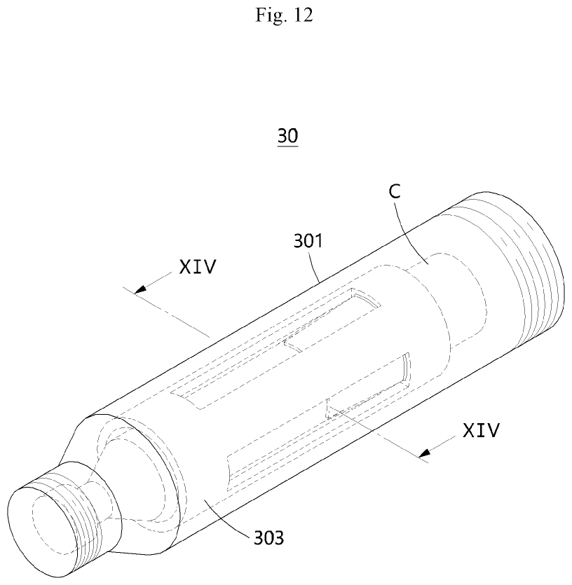

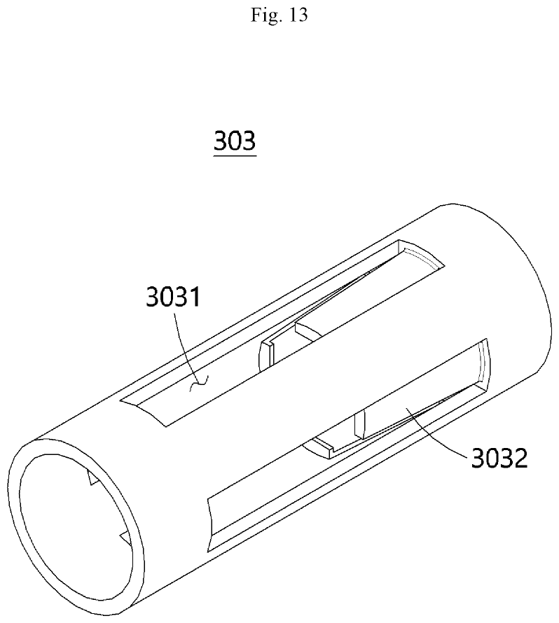

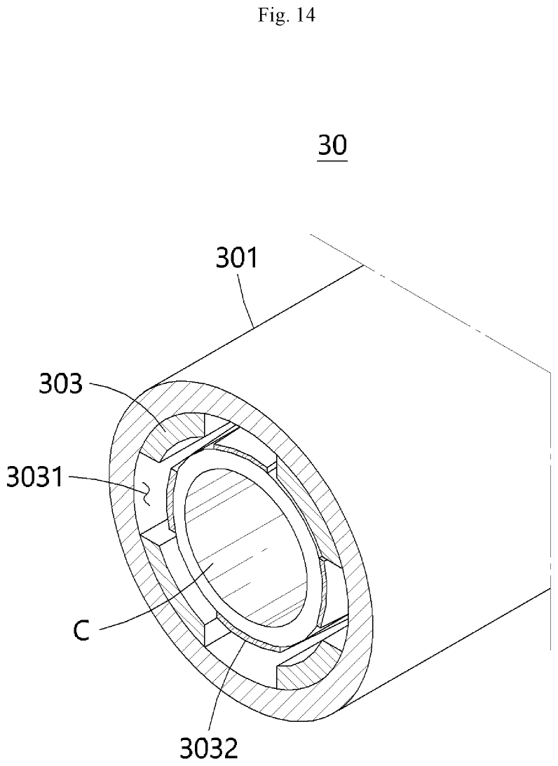

FIG. 12 is a perspective view illustrating a cartridge holder according to an embodiment. FIG. 13 is a perspective view illustrating a cartridge guide according to an embodiment. FIG. 14 is a perspective cross-sectional view taken along line XIV-XIV of FIG. 12.

Referring to FIGS. 12 to 14, according to an embodiment, the cartridge holder 30 may include a holder case 301, a cartridge guide 303, and a cartridge support.

The cartridge holder 30 may include an insertion space formed therein along the lengthwise direction thereof.

A cross section of the cartridge holder 30, perpendicular to the lengthwise direction, may have a ring shape.

The inner diameter of the cartridge holder 30 may be larger than the outer diameter of the medicine cartridge C.

The cartridge guide 303 may be inserted to the insertion space of the holder case 301 and may receive the medicine cartridge C thereinside.

A cross section of the cartridge guide 303, perpendicular to the lengthwise direction, may have a ring shape.

In other words, at the cross section view perpendicular to the lengthwise direction, the cartridge guide 303 may be positioned between the inner circumferential surface of the holder case 301 and the outer circumferential surface of the medicine cartridge C.

The outer diameter of the cartridge guide 303 may be identical to the inner diameter of the holder case 301.

The cartridge guide 303 may include a plurality of guide slots 3031 formed through the surface along the lengthwise direction thereof.

The plurality of guide slots 3031 may be arranged at equal intervals at the cross section view.

The cartridge support may include a plurality of support members 3032 connected to the cartridge guide 303 to be individually placed in the plurality of guide slots 3031.

The plurality of support members 3032 may move in the radial direction of the cartridge support while supporting the outer surface of the medicine cartridge C.

The support members 3032 may apply an external force to the outer surface of the medicine cartridge C by elastic force, thereby aligning the position of the medicine cartridge C in the cartridge guide 303.

While the medicine cartridge C is inserted in the insertion space, the support members 3032 may store elastic energy as going away from the center of the cross section of the holder case 301 and apply an external force to the outer surface of the medicine cartridge C by the stored elastic energy.

By the structure, in the case where the diameter of the medicine cartridge C is smaller than the diameter of the holder case 301, i.e., even when the size of the insertion space of the holder case 301 is larger as compared with the size of the medicine cartridge C, the cartridge guide 303 may stably align the position of the medicine cartridge C.

The cartridge guide 303 may fill the space between the medicine cartridge C and the holder case 301, thereby minimizing a movement of the medicine cartridge C in the insertion space.

Further, the outer surface of the medicine cartridge C may be supported via the cartridge support connected with the cartridge guide 303, allowing the medicine cartridge C to be fixed in the aligned position, i.e., the position in which the centers of the cross sections of the medicine cartridge C and the holder case 301 match each other.

Although the disclosure is shown and described in connection with embodiments, it will be easily appreciated by one of ordinary skill in the art that various changes or modifications may be made without departing from the scope of the disclosure.

For example, although the techniques described herein are performed in a different order from those described herein and/or the components of the above-described structure or device are coupled, combined, or assembled in a different form from those described herein, or some components are replaced with other components or equivalents thereof, a proper result may be achieved.

What is claimed is:

1. A cartridge holder to which a medicine cartridge is coupled, the cartridge holder comprising:
a holder case having an insertion space in which a cartridge is inserted, a cross section of the holder case, perpendicular to a lengthwise direction thereof, having a ring shape; and
a cartridge support connected to the holder case, the cartridge support supporting an outer surface of the medicine cartridge inserted in the insertion space to fix the medicine cartridge.

2. The cartridge holder of claim 1, wherein the cartridge support allows a cross sectional center of the medicine cartridge to match a cross sectional center of the holder case and aligns the medicine cartridge, with the medicine cartridge placed in the insertion space.

3. The cartridge holder of claim 1, wherein the cartridge support includes a support member disposed between an inner circumferential surface of the holder case and an outer circumferential surface of the medicine cartridge, the support member applying an external force to the outer surface of the medicine cartridge.

4. The cartridge holder of claim 3, wherein at cross section view perpendicular to the lengthwise direction of the holder case, the support member protrudes from the inner circumferential surface of the holder case to the cross sectional center of the holder case and contacts the medicine cartridge to adjust a distance from the cross sectional center of the holder case.

5. The cartridge holder of claim 4, wherein the holder case includes a holder slot formed through a surface thereof, and wherein the support member is connected to the holder case to be positioned in the holder slot.

6. The cartridge holder of claim 4, wherein a plurality of support members are connected to the holder case, and wherein at cross section view perpendicular to the lengthwise direction of the holder case, the plurality of support members are spaced apart from each other at equal intervals.

7. The cartridge holder of claim 3, further comprising a cartridge guide inserted in the insertion space and surrounding an outer circumferential surface of the medicine cartridge, wherein the support member is connected to the cartridge guide.

8. The cartridge holder of claim 7, wherein the cartridge guide includes a plurality of guide slots formed through a surface thereof and arranged at equal intervals at cross section view perpendicular to the lengthwise direction thereof, wherein a plurality of support members are placed in the plurality of guide slots, and wherein each of the support members applies an external force in a direction of aligning the medicine cartridge via an elastic force.

9. The cartridge holder of claim 2, wherein the holder case includes an insertion groove formed in an outer surface along a circumferential direction thereof and a plurality of through holes arranged at equal intervals along the insertion groove and formed through the holder case, and wherein the cartridge support includes a ring-shaped ring member fitted to the insertion groove and a plurality of support protrusions connected to the ring member and protruding to a receiving space of the holder case via the through holes.

10. The cartridge holder of claim 9, wherein the support protrusions are formed of a compressible elastic material, the support protrusions applying an elastic force to the medicine cartridge, with the medicine cartridge placed in the receiving space.

11. A medicine injection device, comprising:
a cartridge holder including a holder case including an insertion space in which a medicine-containing medicine cartridge is inserted and a cartridge support placed in the insertion space and applying an external force to an outer surface of the medicine cartridge to thereby align a position of the medicine cartridge;
a needle unit including a needle for injecting the medicine to a user's body; and
a driver connected with the cartridge holder and applying a pressure to the medicine cartridge to discharge the medicine from the medicine cartridge to the needle.

12. The medicine injection device of claim 11, wherein the cartridge support includes a support member to apply an external force to the medicine cartridge to allow a cross sectional center of the medicine cartridge to be positioned coaxially with a cross sectional center of the holder case.

13. The medicine injection device of claim 12, wherein while the medicine cartridge is inserted in the insertion space, the support member stores elastic energy as going away from the cross sectional center of the holder case and applies the external force to the outer surface of the medicine cartridge by the stored energy.

14. The medicine injection device of claim 12, wherein a plurality of support members are formed at equal intervals at cross section view of the medicine cartridge, and wherein the support members contact the outer surface of the medicine cartridge.
